(19) United States
(12) Patent Application Publication
(10) Pub. No.: US 2014/0348884 A1
(43) Pub. Date: Nov. 27, 2014

(54) SHAMPOO COMPOSITION WITH ASSOCIATIVE THICKENERS

(71) Applicant: The Procter & Gamble Company, Cincinnati, OH (US)

(72) Inventors: Jennifer Elaine Hilvert, Cincinnati, OH (US); Denise Christine Winstel, Crescent Springs, KY (US)

(73) Assignee: The Procter & Gamble Company, Cincinnati, OH (US)

(21) Appl. No.: 13/899,950

(22) Filed: May 22, 2013

(51) Int. Cl. A61K 8/84; A61Q 19/00; A61K 8/891; A61Q 5/00

(52) U.S. Cl. CPC A61K 8/84; A61Q 5/002; A61Q 19/00; A61K 8/891
USPC 424/401; 424/70.17; 424/78.02; 424/70.12

(57) ABSTRACT

A shampoo composition with improved in use hair feel, comprising associative thickeners and liquid crystals formed from anionic surfactants and polyDADMAC and wherein the liquid crystals have a particle size greater than about 1 micrometer.

### FIELD OF THE INVENTION

A shampoo composition comprising associative thickeners and liquid crystals to improve in use feel of the product.

### BACKGROUND OF THE INVENTION

Hair can suffer damage from a number of sources, such as environmental exposure to ultraviolet radiation and chlorine, chemical treatment, i.e., bleaching, coloring, perming, as well as mechanical influences, i.e., prolonged use of heated styling appliances.

Various hair products can be used to provide a benefit to damaged hair such as post-shampoo application of hair conditioners (including leave-on or rinse-off products); and hair conditioning shampoos, which cleanse and condition the hair. Liquid crystal technology formed from polydiallyldimethylammonium chloride (polyDADMAC) (INCI name Polyquaternium 6) and sulfate surfactants are effective for enhancing benefits in conditioning shampoos. The liquid crystals increase silicone deposition on hair by reducing the substrate's surface energy. In addition, it protects oxidatively colored hair from fading over time. However, this beneficial effect can be associated with a consumer undesirable draggy feel on the hair during product application, which results from the resistance between the skin and hair surfaces during use of the hair care product.

Therefore, it is desired to have a consumer desirable hair product which can deliver the benefits of liquid crystal technology (including increased deposition of silicones), without the in use consumer negative of resistance between the skin and hair surfaces.

The current hair care product incorporates associative thickeners into a liquid crystal containing conditioning shampoo resulting in a formula texture with a lubricious cushion between hands and hair that is sustained throughout the lathering process. This substantive cushion insures that resistance between the skin and hair surfaces are minimized and that the ability to spread and effectively distribute the formula across hair surfaces is delivered. In doing so, a silky hair feel is delivered in use.

### BRIEF DESCRIPTION OF THE DRAWINGS

While the specification concludes with the claims particularly pointing out and distinctly claiming the invention, it is believed that the present invention will be better understood from the following description taken in conjunction with the accompanying drawings in which:

FIG. 1 is a graph of sensory analysis data.

FIG. 2 is DAT and microscopy data.

### SUMMARY OF THE INVENTION

A shampoo composition comprising from about 5% to about 20% of one or more detersive surfactants, by weight of said shampoo composition; from about 0.05 to about 3% associated thickeners; from about 0.05 to about 0.5% polyquaternium 6; and water. Additionally, wherein the composition comprises liquid crystals having a particle size of greater than about 1 micrometer.

### DETAILED DESCRIPTION OF THE INVENTION

A shampoo composition which has lyotropic liquid crystals of a particular size in combination with associative thickeners are particularly useful in treating damaged hair.

A liquid crystalline state exists structurally between the solid crystalline phase and the liquid phase (i.e. an intermediate between the three dimensionally ordered crystalline state and the completely disordered liquid state).

The term “liquid crystal”, as used herein, means a material having phases that are ordered and/or crystalline in only one or two of their three possible orthogonal directions and are disordered (random and/or liquid-like) in the other dimensions.

The term “lyotropic”, as used herein, means that the ordering effects of a material are induced by changing both its concentration and temperature.

The term “nonvolatile” refers to any material having little or no significant vapor pressure under ambient conditions, and a boiling point under one atmosphere (atm) at least about 250° C. The vapor pressure under such conditions is typically less than about 0.2 mm.

The term “water soluble”, as used herein, means that the polymer is soluble in water in the present composition. In general, the polymer should be soluble at 25° C. at a concentration of 0.1% by weight of the water solvent, at 1%, at 5%, and/or at 15%.

The term “damaged hair”, as used herein, includes moderately damaged hair, as available from International Hair Importers & Products Inc. under the code PGMDST, and hair of a condition similar to PGMDST hair, bleached hair, permed hair, and color-treated hair.

### Shampoo Composition

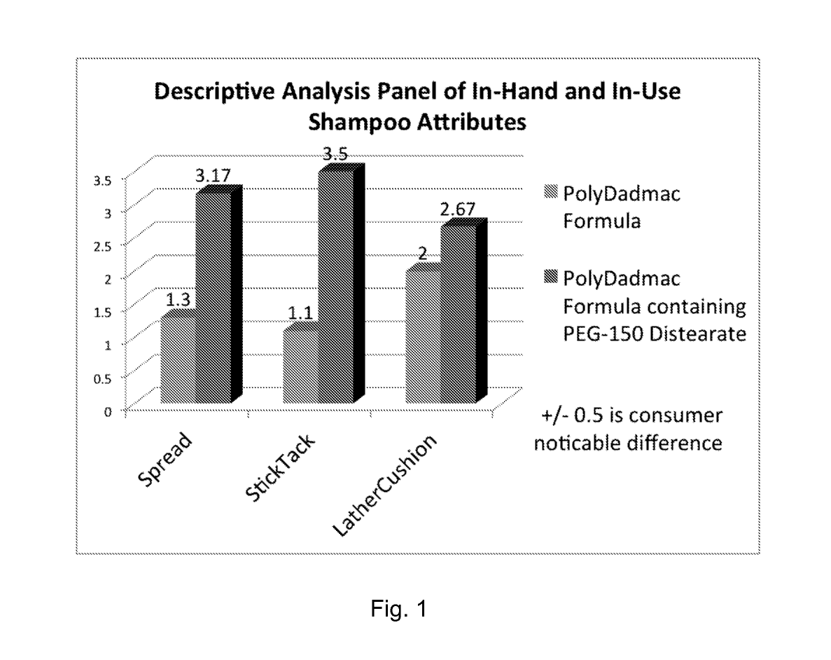

Liquid crystal technology formed between polydiallyldimethylammonium chloride (polyDADMAC) and sulfate surfactants is effective for enhancing benefits in conditioning shampoos. It increases silicone deposition on hair by reducing the substrate's surface energy. In addition, it protects oxidatively colored hair from fading over time (see US Publication 2009/0053165). However the use of polyDADMAC in hair care products can be associated with a draggy feel on the hair during product application (i.e. during the hair washing process), which is not desirable for consumers. The inventive composition mitigates this draggy feel by combining liquid crystal technology with associative thickeners. In one embodiment the associative thickener is polyethyleneglycol distearate. This combination of polyDADMAC and associative thickener achieves good hair conditioning benefits with good in-use feel. This benefit can be demonstrated by a reduction of spread, reduction in tack and increased lather cushion observed by a sensory analysis panel (See FIG. 1 for data).

Additionally, controlling the particle size of the liquid crystal structures can result in the maximum conditioning and in-use experience benefits from the technology. It was observed that particles above about 1 micrometer maximizes the benefit. The particle size can be controlled by adjusting the viscosity of the composition during manufacturing.

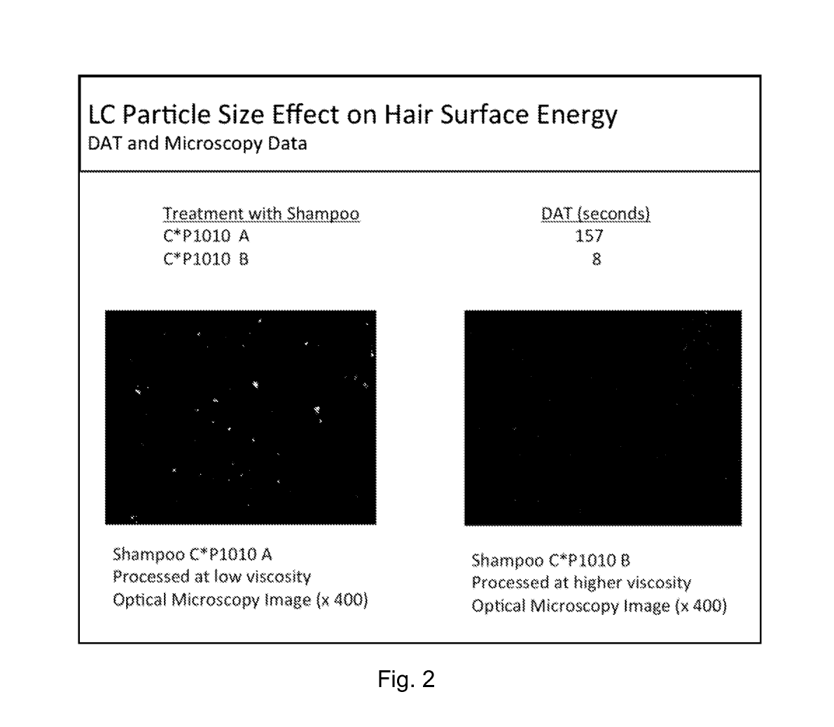

In addition to the in use benefit, hair treated with shampoo having liquid crystals with a particle size of about 1 micrometer or larger resulted in hair with lower surface energy (i.e. more hydrophobic surface) than hair treated with shampoo of the same composition but containing submicron (below about 1 micrometer) liquid crystal particles. The surface energy is shown by measuring the time required for a droplet of water to spread on a hair switch (DAT method). Larger time required for the water droplet to spread on hair switch indicates lower surface energy. The corresponding results are provided in FIG. 2.

In one embodiment the product viscosity (measured via Brookfield rotational viscometer) is in the range of about 1,000-15,000 cP, and in another embodiment in the range of about 2,000-8,000 cP.

A. Lyotropic Liquid Crystals

Lyotropic liquid crystals are formed by combining the synthetic cationic polymers described herein with the anionic detersive surfactant component of the shampoo composition. The synthetic cationic polymer has a relatively high charge density. Suitable cationic polymers will have cationic charge densities of at least about 0.4 meq/gm, at least about 0.0.6 meq/gm, but also less than about 7 meq/gm, and less than about 5 meq/gm, at the pH of intended use of the shampoo composition, which pH will generally range from about pH 3 to about pH 9, and/or between about pH 4 and about pH 8. It should be noted that some synthetic polymers having a relatively high cationic charge density do not form lyotropic liquid crystals, primarily due to their abnormal linear charge densities. Such synthetic cationic polymers are described in WO 94/06403 to Reich et al. The synthetic polymers described herein can be formulated in a stable shampoo composition that provides improved conditioning performance, in particular with respect to damaged hair. In some embodiments, the synthetic cationic polymer can be formed from

i) one or more cationic monomer units, and optionally

ii) one or more momomer units bearing a negative charge, and/or

iii) a nonionic momomer,

wherein the subsequent charge of the copolymer is positive. The ratio of the three types of monomers is given by “m”, “p” and “q” where “m” is the number of cationic monomers, “p” is the number of momomers bearing a negative charge and “q” is the number of nonionic momomers.

When used, the cationic polymers herein are soluble in a complex coacervate phase in the composition formed by the cationic polymer and the anionic, amphoteric and/or zwitterionic detersive surfactant component described hereinbefore. Complex coacervates of the cationic polymer can also be formed with other charged materials in the composition.

Techniques for analysis of formation of complex coacervates are known in the art. For example, microscopic analyses of the compositions, at any chosen stage of dilution, can be utilized to identify whether a coacervate phase has formed. Such coacervate phase will be identifiable as an additional emulsified phase in the composition. The use of dyes can aid in distinguishing the coacervate phase from other insoluble phases dispersed in the composition.

B. Anionic Surfactant Component

The shampoo compositions for treating damaged hair comprise an anionic detersive surfactant component to provide cleaning performance to the composition and to aid in formation of the lyotropic liquid crystalline phase. The anionic surfactant component comprises an anionic detersive surfactant, and optionally, a zwitterionic and/or amphoteric detersive surfactant, which has an attached group that is anionic at the pH of the composition. Such surfactants should be physically and chemically compatible with the essential components described herein or should not otherwise unduly impair product stability, aesthetics, or performance.

Suitable anionic detersive surfactant components include those which are known for use in hair care or other shampoo cleansing compositions. The concentration of the anionic surfactant component generally ranges from about 5% to about 50%, from about 8% to about 30%, from about 10% to about 25%, and from about 12% to about 20%, by weight of the composition.

Suitable anionic detersive surfactants for use in the shampoo compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, disodium laureth sulfosuccinate, disodium laureth-3 sulfosuccinates, dioctyl sodium sulfosuccinate, and combinations thereof.

Suitable amphoteric or zwitterionic detersive surfactants for use in the shampoo composition herein include those which are known for use in hair care or other personal care cleansing compositions and those which contain a group that is anionic at the pH of the shampoo composition. The concentration of such amphoteric detersive surfactants ranges from about 0.5% to about 20%, and from about 1% to about 10% by weight of the composition. Non-limiting examples of suitable zwitterionic or amphoteric surfactants are described in U.S. Pat. Nos. 5,104,646 and 5,106,609.

The shampoo compositions may further comprise additional surfactants for use in combination with the anionic detersive surfactant component described hereinbefore. Suitable optional surfactants include nonionic surfactants, cationic surfactants, and combinations thereof. Any such surfactant known in the art for use in hair or personal care products may be used, provided that the optional additional surfactant is also chemically and physically compatible with the essential components of the shampoo composition or does not otherwise unduly impair product performance, aesthetics or stability. The concentration of optional additional surfactants in the shampoo composition may vary with the cleansing or lather performance desired, the optional surfactant selected, the desired product concentration, the presence of other components in the composition, and other factors well known in the art.

Non limiting examples of other anionic, zwitterionic, amphoteric, or optional additional surfactants suitable for use in the shampoo compositions are described in U.S. Pat. Nos. 3,929,678; 2,658,072; 2,438,091; and 2,528,378.

C. Associated Thickeners

Another class of thickeners along with conventional thickeners is associative thickeners. This class contains polymers which modify the rheology of a fluid through associative interactions between polymer chains, the dispersed phase, and the medium. Unlike conventional thickeners, associative thickeners are often times lower molecular weight polymers containing both hydrophilic and hydrophobic regions. The hydrophobic regions are then able to associate with the hydrophobic moieties while the hydrophilic regions are able to associate with the hydrophilic moieties. This can lead to a network formed within a mixture leading to high viscosities and unique rheological properties.

There are various types of associative thickening polymers, such as hydrophobically modified hydroxyethyl celluloses, hydrophobically modified polypolyacrylates, hydrophobically modified polyacrylic acids, hydrophobically modified polyacrylamides, and hydrophobically modified polyethers.

The class of hydrophobically-modified polyethers include numerous members such as PEG-120-methylglucose dioleate, PEG-N(40 or 60) sorbitan tetraoleate, PEG-150 pentaerythrityl tetrastearate, PEG-55 propylene glycol oleate and PEG-150 distearate. Typically these materials have a hydrophobe, non-limiting examples include cetyl, stearyl, oleayl and combinations thereof, and a hydrophilic portion of repeating ethylene oxide groups with repeat units from 10-300, in an embodiment, from 30-200, and in a further embodiment from 40-150.

The level of associated thickeners, such as PEG-150 distearate, is from about 0.05 to about 3%, from about 0.05 to about 1.5%, from about 0.25 to about 1.5%, and from about 0.25 to about 3%, by weight of the shampoo composition.

D. Synthetic Cationic Polymer

The concentration of the cationic polymers ranges from about 0.025% to about 5%, from about 0.05% to about 3%, and from about 0.1% to about 1%, by weight of the shampoo composition. In one embodiment the cationic polymer is PolyDADMAC (specifically polyquaternium 6) at a level of from about 0.05-0.5%, and in another embodiment from about 0.01-0.25%.

The cationic polymers have a cationic charge density of from about 2 meq/gm to about 7 meq/gm from about 3 meq/gm to about 7 meq/gm, and from about 4 meq/gm to about 7 meq/gm. In some embodiments, the cationic charge density is about 6.2 meq/gm. The polymers also have a molecular weight of from about 1,000 to about 5,000,000, from about 10,000 to about 2,000,000, and from about 100,000 to about 2,000,000.

In one embodiment, the cationic polymers are water soluble or dispersible, non-crosslinked, synthetic cationic polymers having the following structure:

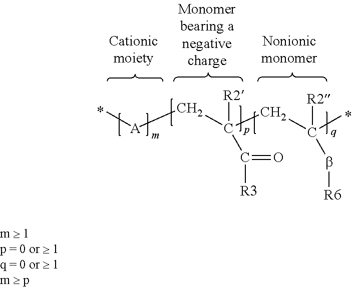

Where A, may be one or more of the following cationic moieties:

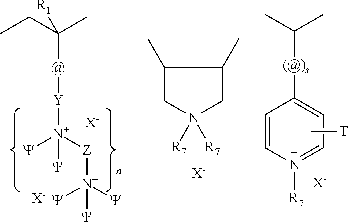

Where @=amido, alkylamido, ester, ether, alkyl or alkylaryl.
Where Y═C1-C2-2 alkyl, alkoxy, alkylidene, alkyl or aryloxy.
Where ψ=C1-C2-2 alkyl, alkyloxy, alkyl aryl or alkyl aryloxy.
Where Z=C1-C2-2 alkyl, alkyloxy, aryl or aryloxy.
Where R1=H, C1-C4 linear or branched alkyl.
Where s=0 or 1, n=0 or ≧1.
Where T and R7=C1-C2-2 alkyl.
Where X—=halogen, hydroxide, alkoxide, sulfate or alkylsulfate.

Where the monomer bearing a negative charge is defined by R2′=H, C1-C4 linear or branched alkyl and R3 as:

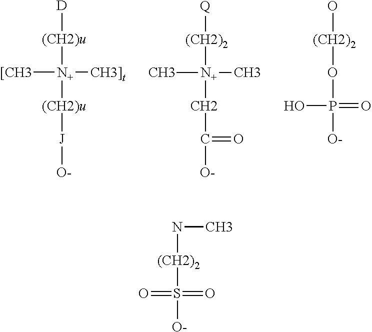

### Where D=O, N, or S.

### Where Q=NH₂ or O.

Where u=1-6.
Where t=0-1.
Where J=oxygenated functional group containing the following elements P, S, C.

Where the nonionic monomer is defined by R2″=H, C1-C4 linear or branched alkyl, R6=linear or branched alkyl, alkyl aryl, aryl oxy, alkyloxy, alkylaryl oxy and β is defined as

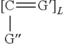

Where G′ and G″ are, independently of one another, O, S or N—H and L=0 or 1.

Examples of cationic monomers include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of cationic monomers include dimethylaminoethyl (meth)acrylate, dimethylaminopropyl (meth)acrylate, ditertiobutylamino ethyl (meth)acrylate, dimethylaminomethyl (meth)acrylamide, dimethylaminopropyl (meth)acrylamide, ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride.

Suitable cationic monomers comprise a quaternary ammonium group of formula —NR₃⁺, wherein R, which is identical or different, represents a hydrogen atom, an alkyl group comprising 1 to 10 carbon atoms, or a benzyl group, optionally carrying a hydroxyl group, and comprise an anion (counter-ion). Examples of anions are halides such as chlorides, bromides, sulphates, hydrosulphates, alkylsulphates (for example comprising 1 to 6 carbon atoms), phosphates, citrates, formates, and acetates.

Suitable cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride.

Additional cationic monomers include trimethyl ammonium propyl (meth)acrylamido chloride.

Examples of monomers bearing a negative charge include alpha ethylenically unsaturated monomers comprising a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Suitable monomers with a negative charge include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

Suitable nonionic monomers include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X—) in association with the synthetic cationic polymers may be any known counterion so long as the polymers remain soluble or dispersible in water, in the shampoo composition, or in a coacervate phase of the shampoo composition, and so long as the counterions are physically and chemically compatible with the essential components of the shampoo composition or do not otherwise unduly impair product performance, stability or aesthetics. Non limiting examples of such counterions include halides (e.g., chlorine, fluorine, bromine, iodine), sulfate and methylsulfate.

E. Optional Ingredients

In accordance with embodiments of the present invention, the shampoo composition may further comprise one or more optional ingredients, including benefit agents Suitable benefit agents include, but are not limited to conditioning agents, silicone emulsions, anti-dandruff actives, gel networks, chelating agents, and, natural oils such as sun flower oil or castor oil. Additional suitable optional ingredients include but are not limited to perfumes, perfume microcapsules, colorants, particles, anti-microbials, foam busters, anti-static agents, rheology modifiers and thickeners, suspension materials and structurants, pH adjusting agents and buffers, preservatives, pearlescent agents, solvents, diluents, anti-oxidants, vitamins and combinations thereof.

Such optional ingredients should be physically and chemically compatible with the components of the composition, and should not otherwise unduly impair product stability, aesthetics, or performance. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein.

The conditioning agent of the compositions of the present invention can be a silicone conditioning agent. The silicone conditioning agent may comprise volatile silicone, non-volatile silicone, or combinations thereof. The concentration of the silicone conditioning agent typically ranges from about 0.01% to about 10%, by weight of the composition, from about 0.1% to about 8%, from about 0.1% to about 5%, and/or from about 0.2% to about 3%. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609, which descriptions are incorporated herein by reference. The silicone conditioning agents for use in the compositions of the present invention can have a viscosity, as measured at 25° C., from about 20 to about 2,000,000 centistokes (“csk”), from about 1,000 to about 1,800,000 csk, from about 50,000 to about 1,500,000 csk, and/or from about 100,000 to about 1,500,000 csk.

The dispersed silicone conditioning agent particles typically have a volume average particle diameter ranging from about 0.01 micrometer to about 50 micrometer. For small particle application to hair, the volume average particle diameters typically range from about 0.01 micrometer to about 4 micrometer, from about 0.01 micrometer to about 2 micrometer, from about 0.01 micrometer to about 0.5 micrometer. For larger particle application to hair, the volume average particle diameters typically range from about 5 micrometer to about 125 micrometer, from about 10 micrometer to about 90 micrometer, from about 15 micrometer to about 70 micrometer, and/or from about 20 micrometer to about 50 micrometer.

Additional material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), incorporated herein by reference.

Silicone emulsions suitable for use in the embodiments of the present invention include, but are not limited to, emulsions of insoluble polysiloxanes prepared in accordance with the descriptions provided in U.S. Pat. No. 4,476,282 and U.S. Patent Application Publication No. 2007/0276087. Accordingly, suitable insoluble polysiloxanes include polysiloxanes such as alpha, omega hydroxy-terminated polysiloxanes or alpha, omega alkoxy-terminated polysiloxanes having a molecular weight within the range from about 50,000 to about 500,000 g/mol. The insoluble polysiloxane can have an average molecular weight within the range from about 50,000 to about 500,000 g/mol. For example, the insoluble polysiloxane may have an average molecular weight within the range from about 60,000 to about 400,000; from about 75,000 to about 300,000; from about 100,000 to about 200,000; or the average molecular weight may be about 150,000 g/mol. The insoluble polysiloxane can have an average particle size within the range from about 30 nm to about 10 micron. The average particle size may be within the range from about 40 nm to about 5 micron, from about 50 nm to about 1 micron, from about 75 nm to about 500 nm, or about 100 nm, for example.

The average molecular weight of the insoluble polysiloxane, the viscosity of the silicone emulsion, and the size of the particle comprising the insoluble polysiloxane are determined by methods commonly used by those skilled in the art, such as the methods disclosed in Smith, A. L. The Analytical Chemistry of Silicones, John Wiley & Sons, Inc.: New York, 1991. For example, the viscosity of the silicone emulsion can be measured at 30° C. with a Brookfield viscosimeter with spindle 6 at 2.5 rpm. The silicone emulsion may further include an additional emulsifier together with the anionic surfactant,

Other classes of silicones suitable for use in compositions of the present invention include but are not limited to: i) silicone fluids, including but not limited to, silicone oils, which are flowable materials having viscosity less than about 1,000,000 csk as measured at 25° C.; ii) aminosilicones, which contain at least one primary, secondary or tertiary amine; iii) cationic silicones, which contain at least one quaternary ammonium functional group; iv) silicone gums; which include materials having viscosity greater or equal to 1,000,000 csk as measured at 25° C.; v) silicone resins, which include highly cross-linked polymeric siloxane systems; vi) high refractive index silicones, having refractive index of at least 1.46, and vii) mixtures thereof

1. Organic Conditioning Materials

The conditioning agent of the shampoo compositions of the present invention may also comprise at least one organic conditioning material such as oil or wax, either alone or in combination with other conditioning agents, such as the silicones described above. The organic material can be non-polymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M and mixtures thereof.

2. Emulsifiers

A variety of anionic and nonionic emulsifiers can be used in the shampoo composition of the present invention. The anionic and nonionic emulsifiers can be either monomeric or polymeric in nature. Monomeric examples include, by way of illustrating and not limitation, alkyl ethoxylates, alkyl sulfates, soaps, and fatty esters and their derivatives. Polymeric examples include, by way of illustrating and not limitation, polyacrylates, polyethylene glycols, and block copolymers and their derivatives. Naturally occurring emulsifiers such as lanolins, lecithin and lignin and their derivatives are also non-limiting examples of useful emulsifiers.

3. Chelating Agents

The shampoo composition can also comprise a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties. This term include alkali metal, alkaline earth, ammonium, substituted ammonium (i.e. monoethanolammonium, diethanolammonium, triethanolammonium) salts, esters of chelants having an acidic moiety and mixtures thereof, in particular all sodium, potassium or ammonium salts. The term “derivatives” also includes “chelating surfactant” compounds, such as those exemplified in U.S. Pat. No. 5,284,972, and large molecules comprising one or more chelating groups having the same functional structure as the parent chelants, such as polymeric EDDS (ethylenediaminedisuccinic acid) disclosed in U.S. Pat. No. 5,747,440.

Levels of the EDDS chelant in the shampoo compositions can be as low as about 0.01 wt % or even as high as about 10 wt %, but above the higher level (i.e., 10 wt %) formulation and/or human safety concerns may arise. In an embodiment, the level of the EDDS chelant may be at least about 0.05 wt %, at least about 0.1 wt %, at least about 0.25 wt %, at least about 0.5 wt %, at least about 1 wt %, or at least about 2 wt % by weight of the shampoo composition. Levels above about 4 wt % can be used but may not result in additional benefit.

4. Additional Deposition Polymer

The shampoo composition may further contain an additional cationic polymer to aid in deposition of the silicone oil component and enhance conditioning performance. Concentrations of the cationic polymer in the composition typically range from about 0.01% to about 3%, from about 0.05% to about 2.0%, and from about 0.1% to about 1.0%. Suitable cationic polymers will have cationic charge densities of at least about 0.4 meq/gm, at least about 0.0.6 meq/gm, but also less than about 7 meq/gm, and less than about 5 meq/gm, at the pH of intended use of the shampoo composition, which pH will generally range from about pH 3 to about pH 9, and/or between about pH 4 and about pH 8. Herein, “cationic charge density” of a polymer refers to the ratio of the number of positive charges on the polymer to the molecular weight of the polymer. The average molecular weight of such suitable cationic guars and cellulose polymers will generally be at least about 800,000

Suitable cationic polymers for use in the compositions of the present invention contain cationic nitrogen-containing moieties such as quaternary ammonium or cationic protonated amino moieties. The cationic protonated amines can be primary, secondary, or tertiary amines (secondary or tertiary), depending upon the particular species and the selected pH of the composition. Any anionic counterions can be used in association with the cationic polymers so long as the polymers remain soluble in water, in the composition, or in a coacervate phase of the composition, and so long as the counterions are physically and chemically compatible with the essential components of the composition or do not otherwise unduly impair product performance, stability or aesthetics. Non limiting examples of such counterions include halides (e.g., chloride, fluoride, bromide, iodide), sulfate and methylsulfate.

Non limiting examples of such polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)).

Suitable cationic polymers for use in the composition include polysaccharide polymers, such as cationic cellulose derivatives. Suitable cationic polysaccharide polymers include those which conform to the formula:

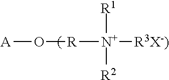

wherein A is an anhydroglucose residual group, such as cellulose anhydroglucose residual; R is an alkylene oxyalkylene, polyoxyalkylene, or hydroxyalkylene group, or combination thereof; R1, R2, and R3 independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R1, R2 and R3) being about 20 or less; and X is an anionic counterion as described in hereinbefore.

Suitable cationic cellulose polymers are salts of hydroxyethyl cellulose reacted with trimethyl ammonium substituted epoxide, referred to in the industry (CTFA) as Polyquaternium 10 and available from Amerchol Corporation (Edison, N.J., USA) in their Polymer LR, JR, JP and KG series of polymers. Other suitable types of cationic cellulose includes the polymeric quaternary ammonium salts of hydroxyethyl cellulose reacted with lauryl dimethyl ammonium-substituted epoxide referred to in the industry (CTFA) as Polyquaternium 24. These materials are available from Amerchol Corporation under the tradename Polymer LM-200.

Suitable cationic guar polymers include cationic guar gum derivatives, such as guar hydroxypropyltrimonium chloride, one example of which includes Jaguar Excel commercially available from Rhodia Corporation. Guar polymers consistent with the present invention are described in U.S. Pat. No. 5,756,720.

Suitable cationic polymers include polymers of sufficiently high cationic charge density to effectively enhance deposition efficiency of the solid particle components described herein. Suitable cationic polymers comprise cationic cellulose polymers and cationic guar derivatives with cationic charge densities of at least about 0.5 meq/gm and less than about 7 meq/gm. Suitable cationic cellulose polymers salts of hydroxyethyl cellulose reacted with trimethyl ammonium substituted epoxide, referred to in the industry (CTFA) as Polyquaternium 10 and available from Amerchol Corp. (Edison, N.J., USA) as Ucare Polymer JR30M with a charge density of 1.32 and a molecular weight of approximately 2,000,000, Ucare Polymer KG30M with a charge density of 1.96 and a molecular weight of approximately 2,000,000, and Ucare Polymer JP with a charge density of 0.7 and a molecular weight of approximately 2,000,000.

The above deposition polymers give good clarity and adequate flocculation on dilution with water during use, provided sufficient electrolyte is added to the formulation. Suitable electrolytes include but are not limited to sodium chloride, sodium benzoate, magnesium chloride, and magnesium sulfate.

5. Aqueous Carrier

The shampoo compositions can be in the form of pourable liquids (under ambient conditions). Such compositions will therefore typically comprise a carrier, which is present at a level of from about 20 wt % to about 95 wt %, or even from about 60 wt % to about 85 wt %. The carrier may comprise water, or a miscible mixture of water and organic solvent, and in one aspect may comprise water with minimal or no significant concentrations of organic solvent, except as otherwise incidentally incorporated into the composition as minor ingredients of other essential or optional components.

The carrier useful in embodiments of the shampoo compositions of the present invention includes water and water solutions of lower alkyl alcohols and polyhydric alcohols. The lower alkyl alcohols useful herein are monohydric alcohols having 1 to 6 carbons, in one aspect, ethanol and isopropanol. Exemplary polyhydric alcohols useful herein include propylene glycol, hexylene glycol, glycerin, and propane diol.

### Product Form

The shampoo compositions of the present invention may be presented in typical shampoo formulations. They may be in the form of solutions, dispersion, emulsions, powders, talcs, encapsulated, spheres, spongers, solid dosage forms, foams, and other delivery mechanisms. The compositions of the embodiments of the present invention may be hair tonics, leave-on hair products such as treatment, and styling products, rinse-off hair products such as shampoos, and treatment products; and any other form that may be applied to hair.

The shampoo composition can have a viscosity of 4,000 cP to 20,000 cP, or from about 6,000 cP to about 12,000 cP, or from about 8,000 cP to about 11,000 cP, measured at 26.6° C. with a Brookfield R/S Plus Rheometer at 2 s⁻¹. cP means centipoises.

### Methods of Use

The compositions described herein are particularly useful in treating damaged hair. Damaged hair includes hair that has been exposed to environmental damage, such as damage from ultraviolet radiation and chlorine, chemical treated hair, i.e., bleached, color-treated, and/or permed hair, as well as mechanically damaged hair, i.e., hair exposed to prolonged use of heated styling appliances. As discussed above, such hair is increasingly hydrophilic (increased surface energy), as compared to virgin hair.

The method of treating damaged hair comprises the steps of contacting damaged hair, which has typically been wetted with water, with an effective amount of the shampoo composition described herein. After contacting the hair with the shampoo composition, the composition is rinsed from the hair. Effective amounts of the shampoo composition generally range from about 1 gm to about 50 gm, and from about 1 gm to about 20 gm. Application to the hair typically includes working the composition through the hair such that most or all of the hair is contacted with the composition.

### Examples

The following examples illustrate the present invention. The exemplified compositions can be prepared by conventional formulation and mixing techniques. It will be appreciated that other modifications of the present invention within the skill of those in the shampoo formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

| Example | 1 | 2 | 3 | 4 | 5 | 6 | 7 | 8 | 9 | 10 |
|---|---|---|---|---|---|---|---|---|---|---|
| Sodium Lauryl Sulfate | 0.00 | 0.00 | 0.00 | 0.00 | 0.00 | 0.00 | 0.00 | 1.50 | 6.00 | 7.00 |
| Sodium Laureth-3 Sulfate | 14.00 | 14.00 | 14.00 | 14.00 | 14.00 | 14.00 | 14.00 | | 10.00 | 8.00 |
| Sodium Laureth-1 Sulfate | | | | | | | | 12.50 | | |
| Cocamidopropyl betaine | 1.50 | 1.50 | 1.50 | 1.50 | 1.50 | 0.75 | 0.75 | 1.50 | | 2.00 |
| Cocomonoethanolamide | 3.00 | 3.00 | 3.00 | 3.00 | 3.00 | 1.50 | 1.50 | 1.00 | 0.85 | |
| Laureth-4 | 0.00 | 0.00 | 0.00 | 0.00 | 0.00 | 0.00 | 0.00 | 0.00 | | |
| Ethylene Glycol Distearate | 1.50 | 1.50 | 1.50 | 1.50 | 1.50 | 1.50 | 1.50 | 1.50 | 1.50 | 1.50 |
| PEG 150 Distearate | 0.25 | 1.25 | 0.25 | 0.25 | 0.25 | 0.75 | 0.75 | 0.50 | 2.00 | 0.25 |
| Stearyl Alcohol | | | | | | | | 1.29 | | |
| Cetyl Alcohol | | | | | | | | 0.71 | | |
| Dimethicone 330M | 1.00 | 1.00 | 0.50 | | | 1.00 | 1.00 | 1.00 | 1.00 | 1.00 |
| Abil Soft AF100 | | 0.50 | | | | | | | | |
| Guar Hydroxypropyl- | | | 0.25 | | | | | | 0.10 | |
| trimonium chloride | | | | | | | | | | |
| Guar Hydroxypropyl- | | | | 0.25 | | | | 0.25 | | |
| trimonium chloride | | | | | | | | | | |
| Guar Hydroxypropyl- | | | | | 0.25 | | | | | 0.40 |
| trimonium chloride | | | | | | | | | | |
| Polyquaternium-10 | | | | | | 0.50 | | | | |
| Polyquaternium-76 | | | | | | | 0.25 | | | |
| Polyquaternium-6 | 0.10 | 0.10 | 0.10 | 0.10 | 0.10 | 0.10 | 0.10 | 0.10 | 0.10 | 0.15 |
| Methyl Paraben | 0.10 | 0.10 | 0.10 | 0.10 | 0.10 | 0.10 | 0.10 | | 0.10 | |
| Sodium Benzoate | 0.50 | 0.50 | 0.50 | 0.50 | 0.50 | 0.50 | 0.50 | 0.50 | 0.50 | |
| Salicylic Acid USP | 0.20 | 0.20 | 0.20 | 0.20 | 0.20 | 0.20 | 0.20 | | 0.20 | |
| Tetrasodium EDTA | 0.15 | 0.15 | 0.15 | 0.15 | 0.15 | 0.15 | 0.15 | 0.15 | 0.15 | 0.15 |
| Tetrahydrate | | | | | | | | | | |
| Trisodium | 0.20 | 0.20 | 0.20 | 0.20 | 0.20 | 0.20 | 0.10 | 0.10 | 0.20 | 0.10 |
| Ethylenediamine | | | | | | | | | | |
| Disuccinate | | | | | | | | | | |
| METHYLCHLORO- | | | | | | | | 0.0005 | | 0.0005 |
| ISOTHIAZOLINONE | | | | | | | | | | |

| | |
|---|---|
| Polyquaternium-10 | U Care Polymer Series: LR400, LR30M, |
| | JR400, JR30M, KG30M |
| Polyquaternium-76 | Polyquaternium- |
| | 76 10% active AM |
| | from Rhodia T |
| Polyquaternium-6 | Mirapol 100S from Rhodia |
| Jaguar C17 | Jaguar C17 from Rhodia |
| Guar 3196 | N-Hance 3196 from Hercules |
| | Aqualon Div |
| C500 | Jaguar C500 from Rhodia |
| Dimethicone 330M | Besil |
| Dimethicone | DM500 |

### Method of Making

The compositions of the present invention, in general, may be made by mixing together at elevated temperature, e.g., about 72.degree. C. water and surfactants along with any solids (e.g. amphiphiles) that need to be melted, to speed mixing into the personal cleansing composition. The ingredients are mixed thoroughly at the elevated temperature and then cooled to ambient temperature. Additional ingredients, including electrolytes, polymers, and particles, may be added to the cooled product. Care must be taken during incorporation of the polyquaternium-6 to ensure the formation of liquid crystals and that the desired particle size is attained. The silicone may be emulsified at room temperature in concentrated surfactant and then added to the cooled product.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A shampoo composition comprising:
a) from about 5% to about 20% of one or more detersive surfactants, by weight of said shampoo composition;
b) from about 0.05 to about 3% associated thickeners;
c) from about 0.05 to about 0.5% polyquaternium 6;
d) water;
wherein said composition comprises liquid crystals having a particle size of greater than about 1 micrometer.

2. The shampoo composition of claim 1, wherein said detersive surfactant is selected from the group consisting of ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate, and mixtures thereof.

3. The shampoo composition of claim 1, wherein said nonionic surfactant is selected from the group consisting of polyoxyethylene alkyl ethers, polyethyleneglycol fatty acid esters, polyoxyethylene castor oil, polyoxyethylene hydrogenated castor oil, polyoxyethylene fatty amides and their monoethanolamine and diethanolamine derivatives, polyethoxylated fatty amines, alkyl polyglucosides, sugar esters, polyglyceryl fatty acid esters, alkyl polyglyceryl ethers, and mixtures thereof.

4. The shampoo composition of claim 1, further comprising a deposition polymer.

5. The shampoo composition of claim 4, wherein said deposition polymer is a cationic polymer.

6. The shampoo composition of claim 5, wherein the cationic polymer is cationic guar.

7. The shampoo composition of claim 5, wherein the shampoo composition comprises from about 0.05% to about 0.6% by weight of the shampoo composition, of cationic guar.

8. The shampoo composition of claim 7, wherein the shampoo composition comprises from about 0.1% to about 0.4% by weight of the shampoo composition, of cationic guar.

9. The shampoo composition of claim 1, wherein said cationic polymer has a molecular weight from about 10,000 to about 10,000,000 and a charge density from about 0.9 meq/g to about 7.0 meq/g.

10. The shampoo composition of claim 9, wherein said charge density is from about 1.0 meq/g to about 3.5 meq/g.

11. A method of treating hair or skin comprising applying to the hair or skin a safe and effective amount of the shampoo composition according to claim 1.

12. The method of treating hair or skin of claim 11, wherein said hair is damaged hair selected from the group consisting of permed hair, oxidatively colored hair, and mechanically damaged hair.

13. The method of treating hair or skin of claim 8, wherein said skin is selected from the group consisting of scalp, face, and body.

14. The shampoo composition of claim 1, wherein the associative thickener is PEG150 Distearate

15. The shampoo composition of claim 14, wherein the associative thickener level if from about 0.05-3.0% by weight of the shampoo composition.

15. The shampoo composition of claim 14, where the PEG150 Distearate from about 0.1 to about 1.5% by weight of the shampoo composition.

16. The shampoo composition of claim 1, further comprising a silicone.
